(19) United States
(12) Patent Application Publication
(10) Pub. No.: US 2026/0125658 A1
(43) Pub. Date: May 7, 2026

(54) FUSION PROTEIN CRTZW AND APPLICATION IN CATALYZING PRODUCTION OF ASTAXANTHIN FROM CAROTENE

(71) Applicant: Yunnan Vitasource Biological Technology Co., LTD., Kunming (CN)

(72) Inventors: Siyu Pan, Kunming (CN); Fei Yang, Kunming (CN); Wei Xiang, Kunming (CN)

(21) Appl. No.: 19/344,674

(22) Filed: Sep. 30, 2025

(30) Foreign Application Priority Data
Nov. 2, 2024 (CN) 202411553439.7

(51) Int. Cl. C12N 9/02; C12P 23/00

(52) U.S. Cl. CPC C12N 9/0077; C12N 9/0083; C12P 23/00; C07K 2319/00; C12Y 114/13129; C12Y 114/15802

(57) ABSTRACT

A fusion protein CrtZW and an application in catalyzing a production of astaxanthin from β-carotene are provided, which relates to the field of astaxanthin synthesis technology. The sequence of the fusion protein CrtZW is shown in SEQ ID NO. 4. The fusion protein can catalyze the synthesis of astaxanthin from β-carotene in one step and is suitable for large-scale production.

### CROSS-REFERENCE TO RELATED APPLICATIONS

This application claims priority to Chinese Patent Application No. 202411553439.7, filed on Nov. 2, 2024, which is hereby incorporated by reference in its entirety.

### SEQUENCE LISTING

The present application contains a sequence listing which was filed electronically in XML format and is hereby incorporated by reference in its entirety. Besides, the XML copy is created on Sep. 30, 2025, is named “PA-2025081291-US-Sequence Listing” and is 15,819 bytes in sizes.

### TECHNICAL FIELD

The present disclosure relates to the field of astaxanthin synthesis technologies, and in particular, to a fusion protein CrtZW and an application in catalyzing a production of astaxanthin from β-carotene.

### BACKGROUND

Astaxanthin is a super strong antioxidant that can be applied in fields such as food, health, beauty, and medicine. The production of astaxanthin mainly includes two methods: chemical synthesis and biosynthesis. The biosynthetic sources of astaxanthin include Haematococcus pluvialis, phaffia rhodozyma, Paracoccus, recombinant Escherichia coli, recombinant lipolytic yeast, etc.

Astaxanthin has two chiral carbon atoms and three conformations: SS, SR, and RR. Only SS has biological activity. In the chemical synthesis process, the SS conformation accounts for only 25% of the total amount and has low biological activity. The biosynthetic process has a long fermentation time, low product concentration, and high subsequent purification costs.

β-carotene can produce astaxanthin through a two-step catalytic reaction: β-carotene produces zeaxanthin under the catalysis of β-carotene hydroxylase, (CrtZ) enzyme, and the zeaxanthin produces astaxanthin under the catalysis of β-carotene ketolase, (CrtW) enzyme; or β-carotene is catalyzed by CrtW enzyme to produce canthaxanthin, and the canthaxanthin is catalyzed by CrtZ enzyme to produce the astaxanthin. The two-step catalytic conversion of β-carotene to produce astaxanthin involves multiple steps, takes a long time, and has a low yield.

Therefore, providing an efficient method for catalyzing the production of astaxanthin from β-carotene is an urgent technical problem that technical personnel in this field need to solve.

### SUMMARY

In view of this, the present disclosure provides a fusion protein CrtZW and its application in catalyzing the production of astaxanthin from β-carotene.

In order to achieve the above objectives, the present disclosure adopts the following technical solution.

A fusion protein CrtZW, where an amino acid sequence of fusion protein CrtZW is SEQ ID NO. 4:

MLWIWNALIVFVTVIGMEVIAALAHKYIMHGWGWGWHLSHHEPRKGAF
EVNDLYAVVFAALSILLIYLGSTGMWPLQWIGAGMTAYGLLYFMVHDGL
VHQRWPFRYIPRKGYLKRLYMAHRMHHAVRGKEGCVSFGFLYAPPLSKL
GGGGSGGGGSGGGGSGGGGSEAAAKEAAAKGGGGSGGGGSGGGGSRQTW
IGLTLAGMIVAGWGSLHVYGVYFHRWGTSSLVIVPAIVAVQTWLSVGLF
IVAHDAMHGSLAPGRPRLNAAVGRLTLGLYAGFRFDRLKTAHHAHHAAP
GTADDPDFYAPAPRAFLPWFLNFFRTYFGWREMAVLTALVLIALFGLGA
RPANLLTFWAAPALLSALQLFTFGTWLPHRHTDQPFADAHHARSSGYGP
VLSLLTCFHFGRHHEHHLTPW, as shown in SEQ ID NO. 4.

As an invention concept similar to the above technical solution, the present disclosure further provides an application of the fusion protein CrtZW in one-step catalytic synthesis of astaxanthin from β-carotene.

Through the above technical solution, it can be seen that the present disclosure obtains the recombinant fusion protein CrtZW by truncating specific sequences of CrtZ enzyme and CrtW enzyme, and then connecting them through a link sequence. This fusion protein can catalyze the synthesis of astaxanthin from β-carotene in one step, which saves the process flow compared to existing technologies and can be applied to industrial production.

### BRIEF DESCRIPTION OF DRAWINGS

One or more embodiments are illustrated with corresponding drawings, which do not constitute a limitation of the embodiments. Elements with the same reference numerals in the drawings represent similar elements, unless otherwise stated, and the figures in the drawings do not constitute a limitation of proportion.

FIG. 1 shows an original structure of CrtZ protein.

FIG. 2 shows an original structure of CrtW protein.

FIG. 3 shows a structure of Fusion Protein.

FIG. 4 shows a comparison of catalytic effects of different enzyme addition methods.

FIG. 5 shows a comparison of catalytic effects of different truncation methods for fusion proteins.

### DESCRIPTION OF EMBODIMENTS

The following provides a clear and complete description of the technical solutions in embodiments of the present disclosure. Obviously, the described embodiments are only a part of the embodiments of the present disclosure, and not all of them. Based on the embodiments of the present disclosure, all other embodiments obtained by those skilled in the art without creative work are within the protection scope oof the present disclosure.

### Example 1 Construction of Fusion Protein

1. an original protein sequence of Pantoea ananatis CrtZ has been truncated, and the original sequence is as follows:

MLWIWNALIVFVTVIGMEVIAALAHKYIMHGWGWGWHLSHHEPRKGAF
EVNDLYAVVFAALSILLIYLGSTGMWPLQWIGAGMTAYGLLYFMVHDGL
VHQRWPFRYIPRKGYLKRLYMAHRMHHAVRGKEGCVSFGFLYAPPLSKL
QATLRERHGARAGAARDAQGGEDEPASGK,

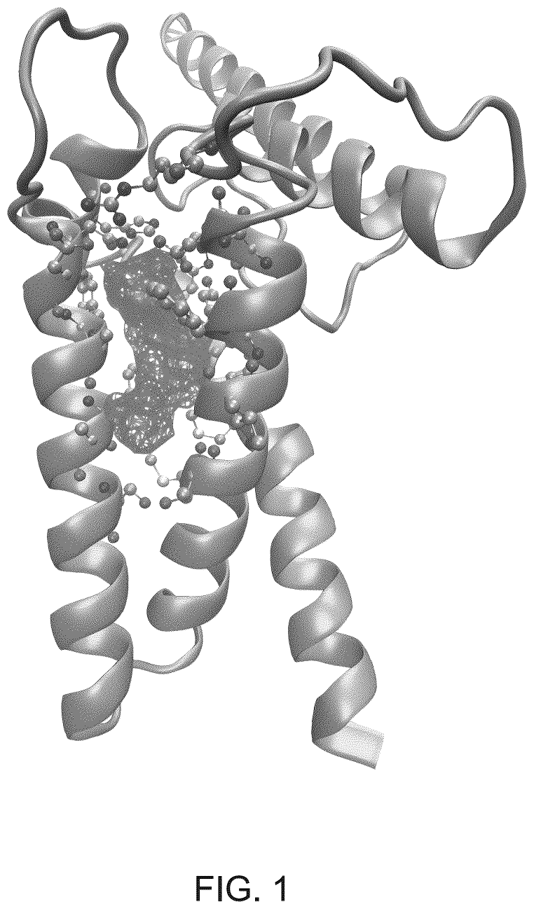

as shown in SEQ ID NO. 1, where the underlined part is the truncated sequence; the original protein structure was constructed using ALPHAFOLD MONOMER V2.0, and a three-dimensional structure of the enzyme was drawn using VMD software. A secondary structure of the enzyme is displayed using the Cartoon drawing method, and purple grid shows the substrate-binding pockets, as shown in FIG. 1.

2. Truncating an original protein sequence of Brevundimonas sp. SD212CrtW, and the original sequence is as follows:

MTAAVAEPRIVPRQTWIGLTLAGMIVAGWGSLHVYGVYFHRWGTSSLVI
VPAIVAVQTWLSVGLFIVAHDAMHGSLAPGRPRLNAAVGRLTLGLYAGF
RFDRLKTAHHAHHAAPGTADDPDFYAPAPRAFLPWFLNFFRTYFGWREM
AVLTALVLIALFGLGARPANLLTFWAAPALLSALQLFTFGTWLPHRHTD
QPFADAHHARSSGYGPVLSLLTCFHFGRHHEHHLTPWRPWWRLWRGES,

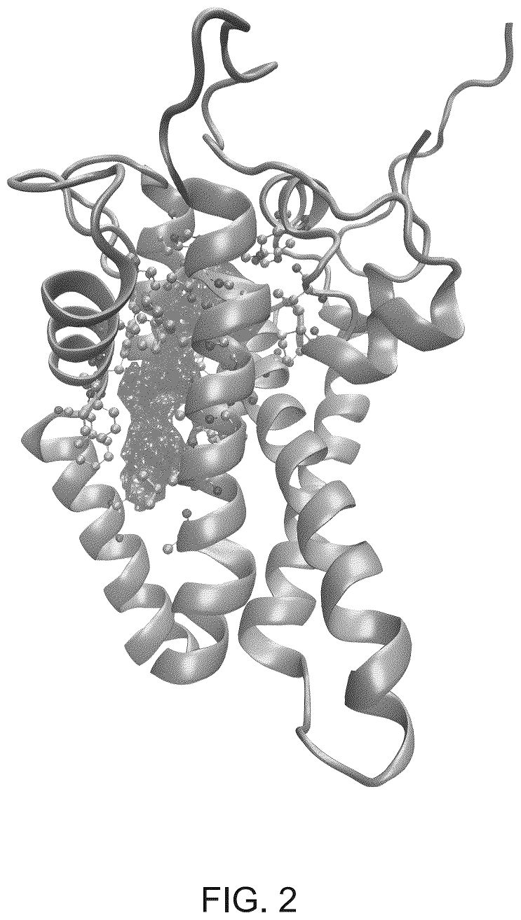

as shown in SEQ ID NO. 2, the underlined part is the truncated sequence, and the structure is constructed using ALPHAFOLD MONOMER V2.0. The three-dimensional structure of the enzyme is drawn using VMD software. The secondary structure of the enzyme is displayed using the Cartoon drawing method, and the purple grid shows the substrate-binding pockets. The structure is shown in FIG. 2.

3. Connecting the truncated sequence with a link sequence:

The Link sequence is as follows:

GGGGSGGGGSGGGGSGGGGSEAAAKEAAAKGGGGSGGGGSGGGGS,
As shown in SEQ ID NO. 3.

Sequence of a fused protein CrtZW is as follows:

MLWIWNALIVFVTVIGMEVIAALAHKYIMHGWGWGWHLSHHEPRKGAF
EVNDLYAVVFAALSILLIYLGSTGMWPLQWIGAGMTAYGLLYFMVHDGL
VHQRWPFRYIPRKGYLKRLYMAHRMHHAVRGKEGCVSFGFLYAPPLSKL
GGGGSGGGGSGGGGSGGGGSEAAAKEAAAKGGGGSGGGGSGGGGSRQTW
IGLTLAGMIVAGWGSLHVYGVYFHRWGTSSLVIVPAIVAVQTWLSVGLF
IVAHDAMHGSLAPGRPRLNAAVGRLTLGLYAGFRFDRLKTAHHAHHAAP
GTADDPDFYAPAPRAFLPWFLNFFRTYFGWREMAVLTALVLIALFGLGA
RPANLLTFWAAPALLSALQLFTFGTWLPHRHTDQPFADAHHARSSGYGP
VLSLLTCFHFGRHHEHHLTPW, as shown in SEQ ID NO. 4.

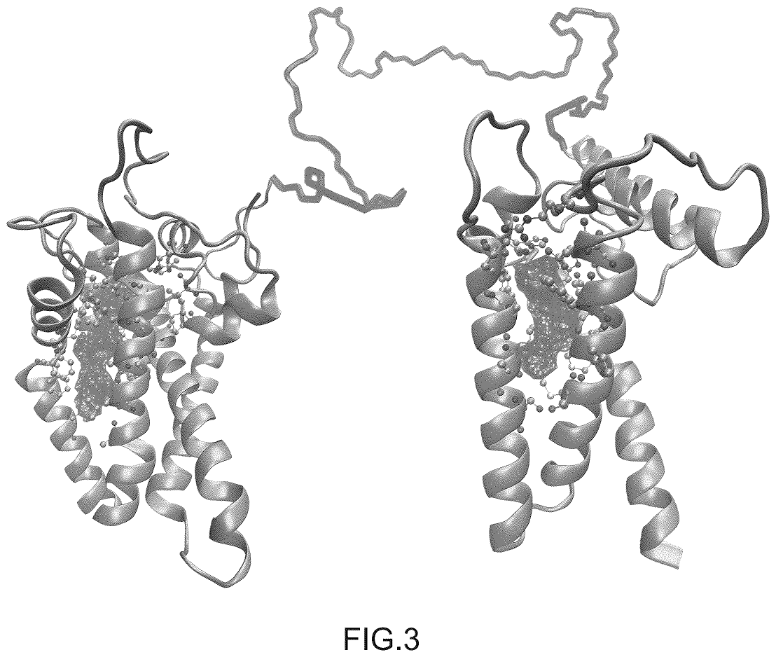

A structure of the fusion protein is shown in FIG. 3. It can be seen from the figure that after appropriate link sequence connection, two proteins can independently form their own structures with little mutual influence.

### Example 2 Comparison of Different Catalytic Methods

Design and synthesize genes based on codon preference of Pichia pastoris, and express recombinant Pantoea ananatis CrtZ, Brevundimonas sp. SD212CrtW, and CrtZW using Pichia pastoris as a chassis cell. Prepare reaction solutions according to different enzyme addition ways, and compare the catalytic effects of different enzymes and addition methods. A configuration of 1 ml reaction solution is as follows: substrate+enzyme solution+buffer solution=400 μL+400 μL+200 μL.

A substrate solution formula is: 0.3 mg/ml β-carotene Tween60 solution (that is polyoxyethylene (20) sorbitan monostearate); a buffer formula is: 0.4 M Tris HCl (pH 7.0), containing 1 mM dithiothreitol, 0.1% v/v Tween 60, 3 mM ATP, 0.5 mM ferrous sulfate, 0.5 mM 2-ketoglutarate, and 5 mM ascorbic acid.

The grouping is as follows:

CrtZ→CrtW: first adding CrtZ and reacting for 3 hours, then adding CrtW and continuing the reaction for 3 hours.

CrtW→CrtZ: first adding CrtW and reacting for 3 hours, then adding CrtZ and continuing the reaction for 3 hours.

CrtZ+CrtW: simultaneously adding CrtZ and CrtW and reacting for 3 hours.

CrtZW: adding CrtZW and reacting for 3 hours.

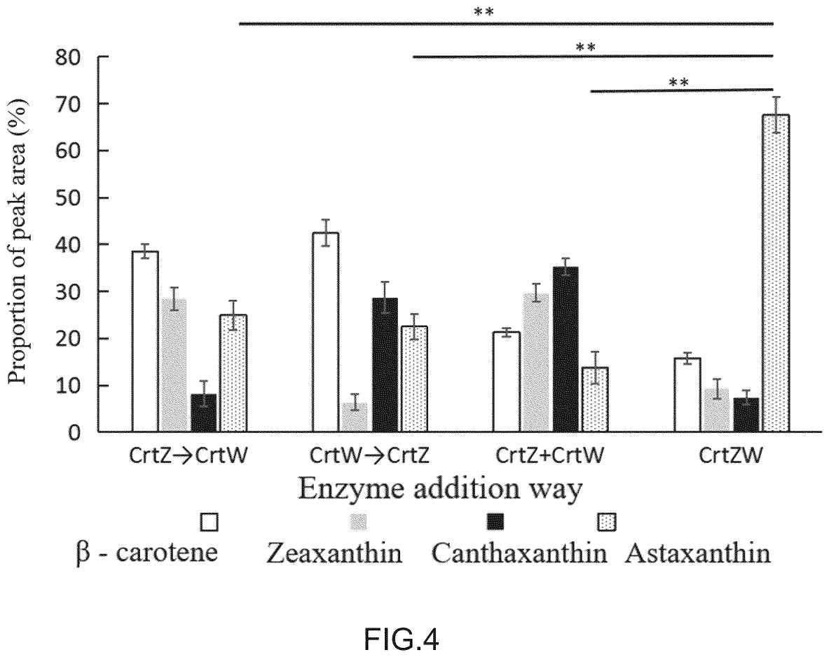

Calculating the astaxanthin content of each group, as shown in FIG. 4.

From FIG. 4, it can be seen that A final astaxanthin content obtained by simultaneously adding two enzymes for the reaction is significantly lower than the catalytic method of sequentially adding two enzymes. The catalytic effect of fusion proteins is significantly better than other catalytic methods.

### Example 3: Catalytic Differences Between Different Links Connecting CrtZ and CrtW

Selecting different link sequences to connect CrtZ and CrtW proteins to form different fusion proteins. Design and synthesize genes based on the codon preference of Pichia pastoris, and express the recombinant fusion protein using Pichia pastoris as the chassis cell. The link sequence is shown in Table 1.

TABLE 1
Comparison of astaxanthin production catalyzed by fusion proteins
connected by different link sequences

| | | Astaxanthin |
|---|---|---|
| Serial | | concentration |
| No. | Link sequence | (mg/ml) |
| Link1 | GGGGSGGGGSGGGGSGGGGSGGGGSGGGGS, such | 0.047 |
| | as, SEQ ID NO. 5 | |
| Link2 | EAAAKEAAAKEAAAKEAAAKEAAAKEAAAK, such | 0.034 |
| | as, SEQ ID NO. 6 | |
| Link3 | GSGSGSGSGSGSGSGSGSGSGSGSGSGSGSGSGSGS | 0.028 |
| | GS, such as, EQ ID NO. 7 | |
| Link4 | GGGSGGGGSGGGSGGGSGGGSGGGSGGGSGGGSG | 0.058 |
| | GGS, such as, EQ ID NO. 8 | |
| Link5 | EAAAKGGGGSEAAAKGGGGSEAAAKGGGGSEAA | 0.012 |
| | AKGGGGS, such as, SEQ ID NO. 9 | |
| Link6 | PTPTPTPTPTPTPTPTPTPTPTPTPTPTPTPTPTPTPTPT | 0.023 |
| | PTPT, such as EQ ID NO. 10 | |
| Link7 | GGGGSEAAAKGGGGSEAAAKGGGGSEAAAK, such | 0.035 |
| | as, SEQ ID NO. 11 | |
| Link8 | GGGGSGGGGSGGGGSGGGGSEAAAKEAAAKGGG | 0.082 |
| | GSGGGGSGGGGS, such as, SEQ ID NO. 3 | |

From Table 1, it can be seen that selecting sequence SEQ ID NO. 3 as the link sequence to connect CrtZ and CrtW results in a best catalytic effect for astaxanthin synthesis. Example 4 Comparison of catalytic effects of different truncation methods

To avoid the protein being too long and forming incorrect folds after fusion that could affect enzyme activity, the two enzymes were truncated separately, retaining only the active site. Connect different truncated proteins through link sequences and recombinant express them in Pichia pastoris to detect the catalytic differences of each fusion protein.

Sequences of different truncated proteins are as follows:

CrtZ C-end truncation
MLWIWNALIVFVTVIGMEVIAALAHKYIMHGWGWGWHLSHHEPRKGAF
EVNDLYAVVFAALSILLIYLGSTGMWPLQWIGAGMTAYGLLYFMVHDGL
VHQRWPFRYIPRKGYLKRLYMAHRMHHAVRGKEGCVSFGFLYAPPLSK
L, as shown in SEQ ID NO. 12.
CrtZ N-end truncation
TVIGMEVIAALAHKYIMHGWGWGWHLSHHEPRKGAFEVNDLYAVVFAAL
SILLIYLGSTGMWPLQWIGAGMTAYGLLYFMVHDGLVHQRWPFRYIPRK
GYLKRLYMAHRMHHAVRGKEGCVSFGFLYAPPLSKLQATLRERHGARAG
AARDAQGGEDEPASGK, as shown in SEQ ID NO. 13.
CrtW C-end truncation
MTAAVAEPRIVPRQTWIGLTLAGMIVAGWGSLHVYGVYFHRWGTSSLVI
VPAIVAVQTWLSVGLFIVAHDAMHGSLAPGRPRLNAAVGRLTLGLYAGF
RFDRLKTAHHAHHAAPGTADDPDFYAPAPRAFLPWFLNFFRTYFGWREM
AVLTALVLIALFGLGARPANLLTFWAAPALLSALQLFTFGTWLPHRHTD
QPFADAHHARSSGYGPVLSLLTCFHFGRHHEHHLTPW, as shown
in SEQ ID NO. 14.
CrtW N-end truncation
RQTWIGLTLAGMIVAGWGSLHVYGVYFHRWGTSSLVIVPAIVAVQTWLS
VGLFIVAHDAMHGSLAPGRPRLNAAVGRLTLGLYAGFRFDRLKTAHHAH
HAAPGTADDPDFYAPAPRAFLPWFLNFFRTYFGWREMAVLTALVLIALF
GLGARPANLLTFWAAPALLSALQLFTFGTWLPHRHTDQPFADAHHARSS
GYGPVLSLLTCFHFGRHHEHHLTPWRPWWRLWRGES, as shown in
SEQ ID NO. 15.

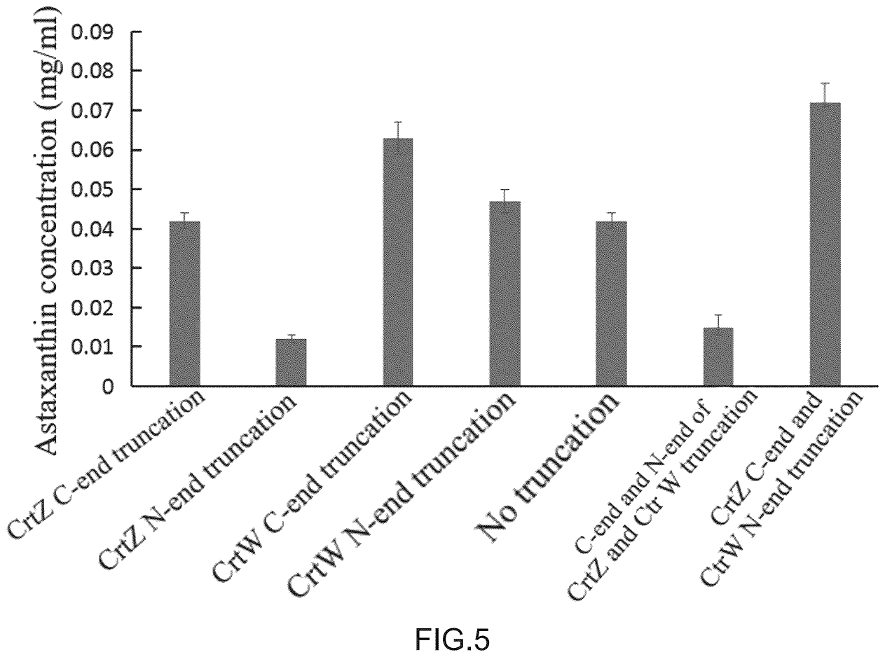

The results are shown in FIG. 5. It can be seen from FIG. 5 that protein truncation has a significant impact on astaxanthin synthesis, but the N-end of CrtZ has a greater negative effect on the fusion protein structure, and truncation can significantly reduce the catalytic effect. The catalytic effect is best when both the C-end of CrtZ and the C and N-ends of CrtW are truncated simultaneously.

Various embodiments in this specification are described in a progressive manner, with each example emphasizing its differences from other examples. The same and similar parts between the examples can be referred to each other.

The above description of the disclosed embodiments enables those skilled in the art to implement or use the present disclosure. Various modifications to these embodiments will be apparent to those skilled in the art, and the general principles defined herein can be implemented in other embodiments without departing from the spirit or scope of the present disclosure. Therefore, the present disclosure will not be limited to the embodiments shown herein, but will be within a widest scope consistent with the principles and features disclosed herein.

What is claimed is:

1. A fusion protein CrtZW, wherein an amino acid sequence of fusion protein CrtZW is SEQ ID NO. 4:
MLWIWNALIVFVTVIGMEVIAALAHKYIMHGWGWGWHLSHHEPRKGAFE
VNDLYAVVFAALSILLIYLGSTGMWPLQWIGAGMTAYGLLYFMVHDGLV
HQRWPFRYIPRKGYLKRLYMAHRMHHAVRGKEGCVSFGFLYAPPLSKLG
GGGSGGGGSGGGGSGGGGSEAAAKEAAAKGGGGSGGGGSGGGGSRQTWI
GLTLAGMIVAGWGSLHVYGVYFHRWGTSSLVIVPAIVAVQTWLSVGLFI
VAHDAMHGSLAPGRPRLNAAVGRLTLGLYAGFRFDRLKTAHHAHHAAPG
TADDPDFYAPAPRAFLPWFLNFFRTYFGWREMAVLTALVLIALFGLGAR
PANLLTFWAAPALLSALQLFTFGTWLPHRHTDQPFADAHHARSSGYGPV
LSLLTCFHFGRHHEHHLTPW, as shown in SEQ ID NO. 4.

2. An application of the fusion protein CrtZW according to claim 1 in one-step catalytic synthesis of astaxanthin from β-carotene.


SEQUENCE LISTING (15 sequences)

SEQ ID NO: 1 - protein, 175 aa, synthetic construct
MLWIWNALIVFVTVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 2 - protein, 244 aa, synthetic construct
MTAAVAEPRIVPRQTWIGLTLAGMIVAGWGSLHVYGVYFHRWGTSSLVIVPAIVAVQTWLSVGLFIVAHDAMHGSLAPGRPRLNAAVGRLTLGLYAGFRFDRLKTAHHAHHAAPGTADDPDFYAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLTPWRPWWRLWRGES

SEQ ID NO: 3 - protein, 45 aa, synthetic construct
GGGGSGGGGSGGGGSGGGGSEAAAKEAAAKGGGGSGGGGSGGGGS

SEQ ID NO: 4 - protein, 412 aa, synthetic construct
MLWIWNALIVFVTVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLGGGGSGGGGSGGGGSGGGGSEAAAKEAAAKGGGGSGGGGSGGGGSRQTWIGLTLAGMIVAGWGSLHVYGVYFHRWGTSSLVIVPAIVAVQTWLSVGLFIVAHDAMHGSLAPGRPRLNAAVGRLTLGLYAGFRFDRLKTAHHAHHAAPGTADDPDFYAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLTPW

SEQ ID NO: 5 - protein, 30 aa, synthetic construct
GGGGSGGGGSGGGGSGGGGSGGGGSGGGGS

SEQ ID NO: 6 - protein, 30 aa, synthetic construct
EAAAKEAAAKEAAAKEAAAKEAAAKEAAAK

SEQ ID NO: 7 - protein, 38 aa, synthetic construct
GSGSGSGSGSGSGSGSGSGSGSGSGSGSGSGSGSGSGS

SEQ ID NO: 8 - protein, 37 aa, synthetic construct
GGGSGGGGSGGGSGGGSGGGSGGGSGGGSGGGSGGGS

SEQ ID NO: 9 - protein, 40 aa, synthetic construct
EAAAKGGGGSEAAAKGGGGSEAAAKGGGGSEAAAKGGGGS

SEQ ID NO: 10 - protein, 44 aa, synthetic construct
PTPTPTPTPTPTPTPTPTPTPTPTPTPTPTPTPTPTPTPTPTPT

SEQ ID NO: 11 - protein, 30 aa, synthetic construct
GGGGSEAAAKGGGGSEAAAKGGGGSEAAAK

SEQ ID NO: 12 - protein, 146 aa, synthetic construct
MLWIWNALIVFVTVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKL

SEQ ID NO: 13 - protein, 163 aa, synthetic construct
TVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 14 - protein, 233 aa, synthetic construct
MTAAVAEPRIVPRQTWIGLTLAGMIVAGWGSLHVYGVYFHRWGTSSLVIVPAIVAVQTWLSVGLFIVAHDAMHGSLAPGRPRLNAAVGRLTLGLYAGFRFDRLKTAHHAHHAAPGTADDPDFYAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLTPW

SEQ ID NO: 15 - protein, 232 aa, synthetic construct
RQTWIGLTLAGMIVAGWGSLHVYGVYFHRWGTSSLVIVPAIVAVQTWLSVGLFIVAHDAMHGSLAPGRPRLNAAVGRLTLGLYAGFRFDRLKTAHHAHHAAPGTADDPDFYAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLTPWRPWWRLWRGES